(19) United States
(12) Patent Application Publication
(10) Pub. No.: US 2024/0335331 A1
(43) Pub. Date: Oct. 10, 2024

(54) TUBE OF COMPRESSION STOCKING WITH ZIPPER AND COMPRESSION STOCKING

(71) Applicant: JIAXING MAX HOSIERY CO., LTD., Jiaxing (CN)

(72) Inventor: Yuliang ZHANG, Jiaxing (CN)

(21) Appl. No.: 18/744,793

(22) Filed: Jun. 17, 2024

Related U.S. Application Data
(63) Continuation of application No. PCT/CN2023/131041, filed on Nov. 10, 2023.

(30) Foreign Application Priority Data
Feb. 9, 2023 (CN) 202320206587.6

(51) Int. Cl. A61F 13/08

(52) U.S. Cl. CPC A61F 13/085

(57) ABSTRACT

A tube of compression stocking with zipper includes a tube body and a zipper on the tube body to press the tube body against the leg, the zipper includes two zipper teeth tapes and a slider to make the two zipper teeth tapes mesh with each other, the slider is provided with an open member to be passed through by fingers, the two zipper teeth tapes extend from an end of the tube body with a smaller inner diameter to another end and are arranged in a V-shape, an end of the two zipper teeth tapes with a greater interval is positioned on the end of the tube body with a greater inner diameter, and a difference between a maximum inner diameter of the tube body and a maximum interval of the two zipper teeth tapes is greater than a minimum inner diameter of the tube body.

### CROSS-REFERENCE TO RELATED APPLICATION

This application is a continuation of PCT application serial no. PCT/CN2023/131041, filed on Nov. 10, 2023, which claims the priority and benefit of Chinese patent application serial no. 202320206587.6, filed on Feb. 9, 2023. The entireties of PCT application serial no. PCT/CN2023/131041 and Chinese patent application serial no. 202320206587.6 are hereby incorporated by reference herein and made a part of this specification.

### TECHNICAL FIELD

The present application relates to a technical field of stockings, and more particular, to a tube of compression stocking with zipper and a compression stocking.

### BACKGROUND ART

A compression stocking is a common sport wear when people exercise, a pressure is applied to the leg of a user by the compression stocking, which may possibly reduce or relieve aftereffects such as muscle soreness on the hind legs and so on after the exercise, and reduce the injury of the leg during the exercise.

Some existing compression stockings are provided with zippers around the tubes of the stockings to facilitate wearing. When the leg of the user extends into the tube of stocking, the zipper is then used to firmly press the tube of stocking against the leg surface of the user, for example, a pre-patent named a compression stocking is disclosed with the publication number of CN217743189U.

For the above related existing technology, when the zipper is zipped up, the user generally needs to firmly pinch the relative small slider to move it, however, the hand of the user tends to sweat a lot after the exercise, the fingers are relatively moist, such that the friction between the fingers and the slider is relatively small, so it is difficult for the user to move the zipper after the exercise.

### SUMMARY

To facilitate the user to move the zipper even after the exercise, the present application discloses a tube of compression stocking with zipper and a compression stocking.

In the first aspect, the tube of compression stocking with zipper according to the present application uses the following solution.

The tube of compression stocking with zipper includes a tube body configured to be passed through by a leg of a user, inner diameters of two ends of the tube body are different. The tube of compression stocking with zipper further includes a zipper on the tube body, the zipper is configured to press the tube body against the leg of the user, the zipper includes two zipper teeth tapes and a slider configured to make the two zipper teeth tapes mesh with each other, the slider is provided with an open member configured to be passed through by fingers of the user, the two zipper teeth tapes extend from a first end of the two ends of the tube body with a smaller inner diameter to a second end of the two ends of the tube body with a greater inner diameter and are arranged in a V-shape, an end of the two zipper teeth tapes with a greater interval is positioned on the second end of the two ends of the tube body with the greater inner diameter, and a difference between a maximum inner diameter of the tube body and a maximum interval of the two zipper teeth tapes is greater than a minimum inner diameter of the tube body.

By adopting the above solution, it is not necessary for the user to pinch the slider of the zipper after the exercise, and it is only necessary to extend the fingers into the open member to pull the open member, such that the slider is moved and the process of taking off or replacing of the compression stocking is completed.

Optionally, the tube body is configured with a tube opening in communication with an exterior and an interior of the tube body, the tube opening penetrates through the two ends of the tube body, and the zipper is provided at the tube opening, so as to force two sides of the tube opening to move towards each other.

By adopting the above solution, the two sides of the zipper can be widely separated, such that the leg of the user is easy to extend into the tube body, especially when the tube body is required to cover the thigh of the user.

Optionally, a position of the tube body where the zipper is located is provided with a tension part, and the tension part is bendable.

By adopting the above solution, two sides of the tube body provided with the zipper will not tend to open excessively, which is conducive to zipping the zipper up to press the tube body against the leg of the user.

Optionally, the tension part includes an external component fixed on a position of the tube body where the tube opening is defined.

By adopting the above solution, the other material, such as a cloth and so on, is sewed on the tube opening, to adjust the range in which the tube opening can be opened according to the requirement, thereby better meeting the special requirements of different persons.

Optionally, the tension part includes an expansion zone formed of an area of the tube body between two sides of the zipper.

By adopting the above solution, it is only necessary to set the primary pressure on the leg of the user by the tube body relatively smaller, which is conducive for the user to wear. When the leg of the user is inserted into the tube body, the zipper may be moved, such that the two sides of the tube body where the zipper is located can move towards each other, thereby stretching the tube body, and thus increasing the pressure on the leg of the user by the tube body.

Optionally, an easy-pull component is provided on the first end of the two ends of the tube body close to a foot of the user, the easy-pull component is close to the zipper, a straight line where the easy-pull component is located is consist with a straight line where the zipper is located, and an elastic modulus of the easy-pull component is greater than an elastic modulus of the tube body.

By adopting the above solution, when the zipper is zipped up to press the tube body against the leg surface of the user. With the easy-pull component, the part of the tube body correspondingly in the same straight line as the zipper is difficult to be stretched, such that the corresponding part of the tube body is difficult to be greatly deformed when the user zips the zipper up by a single hand, thereby zipping the zipper up more effectively.

Optionally, the easy-pull component includes a silk or linen.

By adopting the above solution, the elongation under tension of the silk or line is relatively small.

Optionally, the external component includes a polyester fabric.

By adopting the above solution, the external component possesses excellent elasticity, such that the external component doesn't tend to bend while shrinking when the zipper is zipped up, thereby reducing the foreign body sensation felt by the lower leg of the wear.

Optionally, the open member includes a ring.

By adopting the above solution, the fingers of the user may penetrate the open member, such that the open member can be more stably pulled, and an injury of the fingers of the user is not easy to occur with the ring.

In the second aspect, a compression stocking according to the present application adopts the following solution.

The compression stocking is provided with the above tube of compression stocking with zipper, and the first end of the tube body with the smaller inner diameter is provided with a stocking body configured for the user to wear on a foot.

By adopting above solution, the design can be conducted according to the requirement of the user, and the wear of the stocking body is conducive to correct wearing of the tube of compression stocking.

In summary, the present application has at least one of the following beneficial effectives:

It is not necessary for the user to pinch the slider of the zipper after the exercise, and it is only necessary to extend the fingers into the open member to pull the open member, such that the slider is moved, and the process of taking off or replacing of the compression stocking is completed.

The corresponding part of the tube body is difficult to be greatly deformed when the user zips the zipper up by a single hand, thereby zipping the zipper up more effectively.

### BRIEF DESCRIPTION OF THE DRAWINGS

FIG. 1 is a schematic structure view of a tube of compression stocking with zipper according to the present application with an opening;

FIG. 2 is a schematic structure view of the tube of compression stocking with zipper according to the present application without the opening;

FIG. 3 is a schematic structure view of a compression stocking of the present application.

### DETAILED DESCRIPTION

The present application is further described in detail below in combination with the figures.

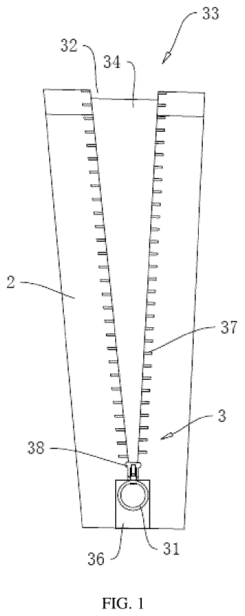

The embodiments of the present application disclose a tube of compression stocking with zipper, referring to FIG. 1, including a tube body 2 which is configured to be sleeved on the leg of the user, the inner diameters of two ends of the tube body 2 are different, to be adapted to positions with different sizes of the leg of the user, which means that an end of the tube body close to the foot of the user has a relatively small diameter. The tube body 2 may only cover the lower leg of the user, or may cover both the lower leg and the thigh of the user according to the requirement. In the present embodiment, the tube body 2 only covers the lower leg of the user. The tube body 2 may be made of a knitted fabric with high elasticity. One side of the tube body 2 is provided with a zipper 3, the zipper 3 forces two sides of the tube body 2 where the zipper 3 is located to move towards each other, such that the tube body 2 is stretched, thereby pressing the tube body 2 against the leg surface of the user. The position of the tube body 2 where the zipper is located is provided with a tension part 33 which is bendable, such that the opening corresponding to the tube body 2 is not easy to expand randomly, when the zipper 3 is pulled open.

Referring to the drawings, the zipper 3 includes two zipper teeth tapes 37 sewed on the tube body 2, the zipper teeth tapes 37 are provided on an end of the tube body with a greater inner diameter, and the zipper teeth tapes are spaced apart from the end of the tube body with a smaller inner diameter. The two zipper teeth tapes 37 are arranged in a V-shape, and the ends of the two zipper teeth tapes 37 with a smaller distance from each other are close to the first end of the tube body 2 with the smaller inner diameter. A slider 38 is slidably connected to the two zipper teeth tapes 37, and the slider 38 is pulled in the length direction of the tube body 2, such that the two zipper teeth tapes 37 are meshed with each other. The difference between the maximum inner diameter of the tube body 2 and the maximum interval between the two zipper teeth tapes 37 is greater than the minimum inner diameter of the tube body 2, such that after the zipper is fully unzipped, the leg of the user can conveniently extend into the tube body 2 through the second end of the tube body 2 with the greater inner diameter.

Referring to FIG. 1, the slider 38 is provided with an open member 31 configured to be inserted in by the finger of the user, the open member 31 may be welded to, hinged to, or integrated with the slider 38, in the present embodiment, the open member 31 is hinged to the slider 38. The open member 31 may be a ring, such that the finger of the user passes through the open member 31, to pull the open member 31 to move the slider 38, thereby pressing the inner wall of the tube body 2 against the leg of the user.

Referring to FIG. 1, a tube opening 32 is defined at the tube body 2 along the length direction thereof, which penetrates an inner wall and outer wall of the tube body 2, and may penetrate two ends or one end of the tube body 2 according to the special requirement. In the present embodiment for representation, the tube opening 32 penetrates the end of the tube body 2 away from the foot of the user. The zipper 3 is provided at two sides of the tube opening 32 along the length direction of the tube opening, such that the tube opening 32 can be closed by the zipper 3. The tension part 33 may be an external component 34 sewed on the two sides of the tube opening 32 along the length direction of the tube opening, in which the external component 34 may be made of a polyester fabric with excellent elasticity.

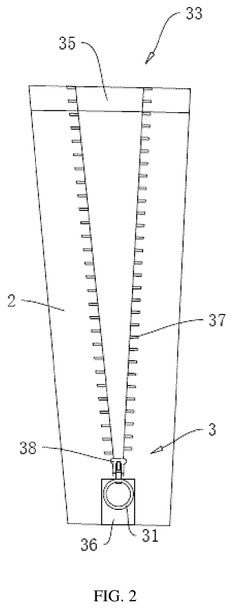

Referring to FIG. 2, in the other embodiment, the tube body 2 is not configured with the tube opening 32, and the circumference of leg section of the user is smaller than the circumference of the tube opening of the tube body 2 when the zipper 3 is not zipped, which is conducive for the user to extend the leg into the tube body 2 conveniently. The tension part 33 may be an expansion zone 35 formed of the area of the tube body 2 between two sides of the zipper 3. When the zipper 3 is zipped, the expansion zone 35 is bended and deformed, by which the circumferences of the sections of the tube body provided with the zipper 3 are reduced, such that the tube body 2 can be firmly pressed against the leg surface of the user.

Referring to FIG. 1, the zipper 3 can be provided on two ends of the tube body 2 according to the requirement, there may be an interval between the zipper 3 and one end of the tube body 2, and there may be an interval between the zipper 3 and each of two ends of the tube body 2. For example, in the present embodiment, when there is the interval between the zipper 3 and the end of the tube body 2 close to the foot of the user, the end of the tube body 2 close to the foot of the user is provided with an easy-pull component 36, which may be made of a fabric which is difficult to be extended, such as a silk or linen. A straight line where the length direction of the easy-pull component 36 is located is consist with a straight line where the length direction of the tube body is located, and the elastic modulus of the easy-pull component 36 is much less than the elastic modulus of the tube body 2, therefore, when the zipper 3 is zipped, the easy-pull component 36 is difficult to be stretched to deform, such that the part of the tube body 2 close to the foot of the user and corresponding to the zipper 3 is difficult to be stretched, therefore, the zipper 3 can be successfully zipped up by a single hand of the user.

In other implementations of the present embodiment, the easy-pull component 36 may be made of a fabric that has better elasticity and is easy to be stretched, while the easy-pull component 36 is sewed with a silk thread, which is difficult to be stretched, along the movement direction of the slider 38 of the zipper 3, such that the easy-pull component 36 is difficult to be stretched similarly.

The implement principle of the tube of compression stocking with zipper is as follows of the embodiments of the present application. The zipper 3 is relatively convenient to be zipped by using the open member 31 and the easy-pull component 36.

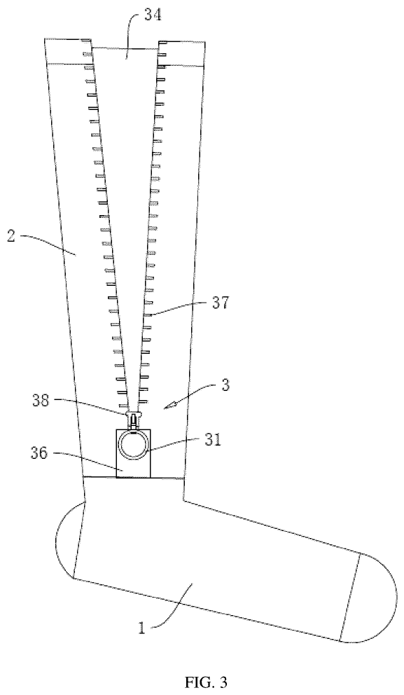

A compression stocking is further disclosed in the present embodiment, which is provided with the above tube of compression stocking. Referring to FIG. 3, a stocking body 1 is integrated with an end of the tube body 2 near the foot of the user, and the user wears the stocking body 1 on the foot.

The implementation principle of the compression stocking is as follows. It can be selected to use the single tube body 2 or use the stocking body 1 in combination with the tube body 2.

The above are the preferred embodiments of the present application, which are not intended to limit the protection scope of the present application. Therefore, all equivalent changes made according to the structure, shape and principle of the present application should be covered within the protection scope of the present application.

### LIST OF REFERENCE SIGNS

1 stocking body
2 tube body
3 zipper
31 open member
32 tube opening
33 tension part
34 external component
35 expansion area
36 easy-pull component
37 zipper teeth tape
38 slider

What is claimed is:

1. A tube of compression stocking with zipper, comprising a tube body configured to be passed through by a leg of a user, inner diameters of two ends of the tube body are different, further comprising a zipper on the tube body, the zipper is configured to press the tube body against the leg of the user, wherein the zipper comprises two zipper teeth tapes and a slider configured to make the two zipper teeth tapes mesh with each other, the slider is provided with an open member configured to be passed through by fingers of the user, the two zipper teeth tapes extend from a first end of the two ends of the tube body with a smaller inner diameter to a second end of the two ends of the tube body with a greater inner diameter and are arranged in a V-shape, an end of the two zipper teeth tapes with a greater interval is positioned on the second end of the two ends of the tube body with the greater inner diameter, and a difference between a maximum inner diameter of the tube body and a maximum interval of the two zipper teeth tapes is greater than a minimum inner diameter of the tube body.

2. The tube of compression stocking with zipper according to claim 1, wherein the tube body is configured with a tube opening in communication with an exterior and an interior of the tube body, and the tube opening penetrates through the two ends of the tube body, and the zipper is provided at the tube opening, so as to force two sides of the tube opening to move towards each other.

3. The tube of compression stocking with zipper according to claim 1, wherein a first position of the tube body where the zipper is located is provided with a tension part, and the tension part is bendable.

4. The tube of compression stocking with zipper according to claim 3, wherein the tension part comprises an external component fixed on a second position of the tube body where a tube opening of the tube body is defined.

5. The tube of compression stocking with zipper according to claim 3, wherein the tension part comprises an expansion zone formed of an area of the tube body between two sides of the zipper.

6. The tube of compression stocking with zipper according to claim 1, wherein an easy-pull component is provided on the first end of the two ends of the tube body close to a foot of the user, the easy-pull component is close to the zipper, a straight line where the easy-pull component is located is consistent with a straight line where the zipper is located, and an elastic modulus of the easy-pull component is greater than an elastic modulus of the tube body.

7. The tube of compression stocking with zipper according to claim 6, wherein the easy-pull component comprises a silk or linen.

8. The tube of compression stocking with zipper according to claim 4, wherein the external component comprises a polyester fabric.

9. The tube of compression stocking with zipper according to claim 1, wherein the open member comprises a ring.

10. A compression stocking provided with the tube of compression stocking with zipper according to claim 1, wherein the first end of the two ends of the tube body with the smaller inner diameter is provided with a stocking body configured for the user to wear on a foot.
